(19) United States
(12) Patent Application Publication
(10) Pub. No.: US 2006/0247319 A1
(43) Pub. Date: Nov. 2, 2006

(54) Preparation of 4-bromotetrahydrofluoreneones

(76) Inventors: Mark A. Huffman, Warren, NJ (US); Xue-Zhi Qin, Lansdale, PA (US); Jonathan D. Rosen, Brooklyn, NJ (US)

Correspondence Address: MERCK AND CO., INC, P O BOX 2000, RAHWAY, NJ, 07065-0907, US

(21) Appl. No.: 11/412,598

(22) Filed: Apr. 26, 2006

Related U.S. Application Data
(60) Provisional application No. 60/676,333, filed on Apr. 29, 2005.

(51) Int. Cl. A61K 31/12; C07C 45/63

(52) U.S. Cl. 514/680; 568/316

(57) ABSTRACT

This invention describes a process for making 4-bromotetrahydrofluorenones, including novel bromination and chlorination steps, and interemediates. Also described are novel tetrahydrofluoreneones.

### BACKGROUND OF THE INVENTION

This invention describes a process for making 4-bromotetrahydrofluorenones. The preparation of tetrahydrofluorenones has been described in the art, see, e.g. Battacharya et al. Angew. Chem. Int. Ed. Engl. 25, 1986, 476. The selective bromination step described herein allows for the formation of 4-bromotetrahydrofluorenones without competing bromination at the reactive 2, 5 and 9 positions. The invention allows for the preparation of highly enantiomerically enriched 4-bromotetrahydrofluorenones by providing for an upgrade of enantiomeric excess without chromatography prior to the final bromination step. The process further provides a novel selective chlorination at the 4-position of indanone intermediates without competing chlorination at the reactive 2-position, and intermediates produced there from.

### SUMMARY OF THE INVENTION

By this invention, there are provided processes for the preparation of compounds of structural formula I: 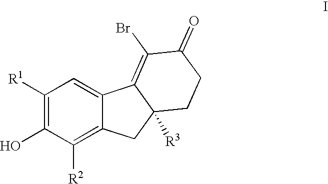
comprising reacting a compound of formula II with a brominating agent 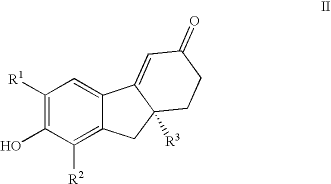
wherein R¹ is halo; R² is halo; and R³ is C₁₋₆ alkyl.

### DETAILED DESCRIPTION OF THE INVENTION

By this invention, there are provided processes for the preparation of compounds of structural formula I: 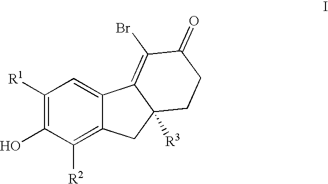
comprising reacting a compound of formula II with a brominating agent 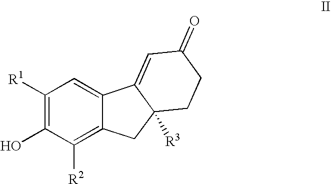
wherein R¹ is halo; R² is halo; and R³ is C₁₋₆ alkyl.

In an embodiment of the invention, the brominating agent is Br₂, NBS or 1,3-dibromo-5,5-dimethylhydantoin. In a class of the embodiment, the brominating agent is Br₂, and the reaction is performed in the presence of a weak base.

In an embodiment of the invention, the weak base is, sodium acetate, potassium acetate, NaHCO₃, KHCO₃, imidazole, or pyridine wherein said imidazole or pyridine bases are optionally substituted with one or two substituents selected from halo, aryl or C₁₋₆ alkyl. In a class of the invention, the weak base is imidazole.

An embodiment of the invention includes a compound of formula I produced by the process disclosed herein. In a class of the embodiment, R¹ is fluoro; R² is chloro; and R³ is butyl. A further class of the embodiment includes crystalline 4-bromo-9a(S)-butyl-8-chloro-6-fluoro-7-hydroxy-1,2,9,9a-tetrahydro-3H-fluoren-3-one.

Another embodiment of the invention includes a process for making a compound of formula III: 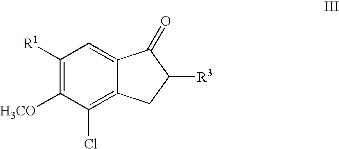
comprising reacting a compound of formula IV with a chlorinating agent 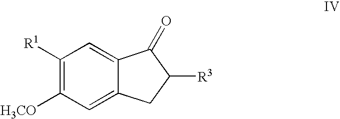
wherein R¹ is halo; and R³ is C₁₋₆ alkyl.

In an embodiment of the invention, the chlorinating agent is NaOCl, Cl₂, t-butylhypochlorite, N-chlorosuccinimide or 1,3-dichloro-5,5-dimethylhydantoin. In a class of the embodiment, an acid is added. In a subclass of the invention, the acid is acetic acid, propionic acid, hydrochloric acid or sulfuric acid.

An embodiment of the invention includes a compound of formula IV wherein R¹ is fluoro and R³ is butyl.

As used herein, “alkyl” is intended to include both branched and straight-chain saturated aliphatic hydrocarbon groups having one to ten carbon atoms unless otherwise specified. For example, C₁₋₁₀, as in “C₁₋₁₀ alkyl” is defined to include groups having 1, 2, 3, 4, 5, 6, 7, 8, 9 or 10 carbons in a linear, branched, or cyclic arrangement. For example, “C₁₋₁₀ alkyl” specifically includes methyl, ethyl, propyl, butyl, pentyl, hexyl, heptyl, octyl, nonyl, decyl, and so on.

As appreciated by those of skill in the art, “halo” or “halogen” as used herein is intended to include chloro, fluoro, bromo and iodo.

The anhydrous solid phase of the free phenol of 4-bromo-9a(S)-butyl-8-chloro-6-fluoro-7-hydroxy-1,2,9,9a-tetrahydro-3H-fluoren-3-one, also known free phenol Form A or novel polymorphic Form A, is characterized by an X-ray powder diffraction (XRPD) pattern (collected using copper Kα radiation) with peaks at 8.6, 21.9, and 25.6 degrees (2θ). The pattern is further characterized by peaks at 17.5, 20.2, and 24.1 degrees (2θ). Additional peaks attributed to Form A are observed at 14.5, 27.2, and 28.0 degrees (2θ).

Differential scanning calorimetry results on free phenol Form A, obtained at a heating rate of 10° C./min in a crimped aluminum pan, indicate a minor endotherm with an extrapolated onset temperature of approximately 172° C. and a major endotherm with an extrapolated onset temperature of approximately 177° C. In an embodiment of the invention, polymorphic Form A of 4-bromo-9a(S)-butyl-8-chloro-6-fluoro-7-hydroxy-1,2,9,9a-tetrahydro-3H-fluoren-3-one is characterized by differential scanning calorimetry, at a heating rate of 10° C./min in a crimped aluminum pan, that exhibits a minor endotherm with an extrapolated onset temperature of about 172° C. In a class of the embodiment, the polymorphic Form A is further characterized by differential scanning calorimetry, at a heating rate of 10° C./min in a crimped aluminum pan that exhibits a major endotherm with an extrapolated onset temperature of about 177° C.

The free phenol Form A is further characterized by single crystal X-ray diffraction. The unit cell of the Form A phase has characteristic dimensions of a=22.2, b=7.4, and c=11.1 angstroms, and characteristic crystallographic angles of α=90, β=111.1, and γ=90 degrees.

The solid phase of the free phenol of 4-bromo-9a(S)-butyl-8-chloro-6-fluoro-7-hydroxy-1,2,9,9a-tetrahydro-3H-fluoren-3-one known as Form B, or novel polymorphic Form B, is obtained by heating Form A to a temperature between approximately 125° C. and 172° C. In this temperature range, the XRPD pattern of Form B is obtained, with characteristic diffraction peaks (collected using copper Kα radiation) at 8.4, 21.3, and 23.6 degrees (2θ). The pattern is further characterized by peaks at 13.8, 27.0, and 29.3 degrees (2θ). Additional peaks attributed to Form B are observed at 14.3, 16.1, 16.8, 17.3, and 25.1 degrees (2θ).

The solid phase of the free phenol of 4-bromo-9a(S)-butyl-8-chloro-6-fluoro-7-hydroxy-1,2,9,9a-tetrahydro-3H-fluoren-3-one known as Form C, or novel polymorphic Form C, is obtained by heating Form A to a temperature of approximately 173° C. and cooling to room temperature. The XRPD pattern (collected using copper Kα radiation) exhibits characteristic peaks at 8.9 and 24.5 degrees (2θ). The pattern is further characterized by peaks at 14.4, 15.1, and 17.0 degrees (2θ). Additional peaks attributed to Form C are observed at 20.0, 20.5, 22.8, and 26.4 degrees (2θ).

In the following schemes and examples below, various reagent symbols and abbreviations have the following meanings:

| | |
|---|---|
| AcOH | Acetic acid |
| AlCl₃ | Aluminum chloride |
| Br₂ | Bromine |
| Cl₂ | Chlorine |
| CF₃Bn—CN—Br | N-(4-trifluoromethylbenzyl)cinchoninium bromide |
| CH₂Cl₂ | Methylene chloride |
| CH₂O | Formaldehyde |
| EtOH | Ethanol |
| H₂SO₄ | Sulfuric acid |
| K₂CO₃ | Potassium carbonate |
| MeOH | Methanol |
| NBS | N-bromosuccinimide |
| NaOCl | Sodium hypochlorite |
| NaOH | Sodium hydroxide |

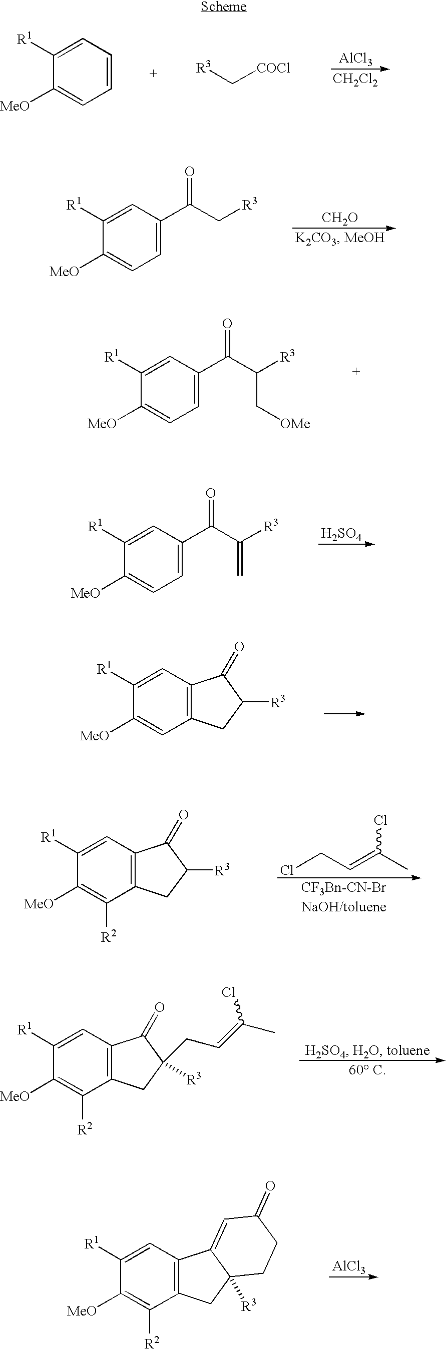

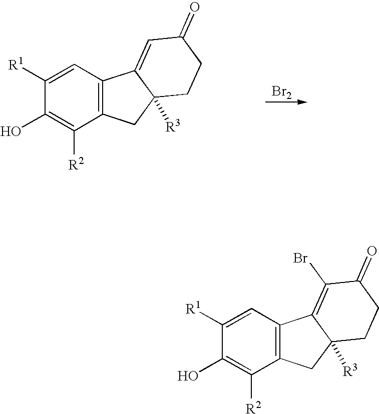

### EXAMPLE 1

### Preparation of 1-(3-Fluoro-4-Methoxy-Phenyl)-Hexan-1-One

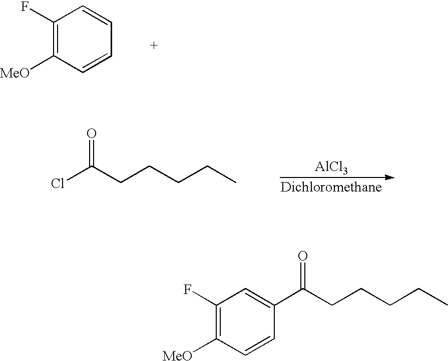

2-Fluoroanisole (3.15 g, 25.0 mmol) is dissolved in dry dichloromethane (25 mL). While stirring the resulting solution under nitrogen, aluminum chloride is added (3.92 g, 29.4 mmol). This solution is cooled with a cold water bath and hexanoyl chloride (3.87 g, 28.8 mmol) is added drop wise such that the internal temperature is maintained at or below 20° C. Upon complete of addition, the reaction is stirred 30 minutes at room temperature.

The dark orange solution is cooled to −10° C. and slowly quenched by the addition of water (25.0 mL) while keeping the internal temperature below 0° C. The mixture is transferred to a separatory funnel, the organic layer (bottom) is collected and the aqueous discarded. The organic solution is then washed with 5N NaOH (12.5 mL) and again with water (12.5 mL).

The crude organic is concentrated by vacuum distillation and diluted with heptane to a final volume of about 20 mL. The slurry is heated until all solids are dissolved and allowed to cool to room temperature. The product crystallizes upon cooling. The slurry is further cooled to 0 to 5° C. for one hour and filtered. The wet cake is washed with cold (0° C.) heptane (2×2.5 mL) and the product dried under vacuum.

### EXAMPLE 2

### Preparation of 1-(3-Fluoro-4-Methoxy-Phenyl)-2-Methylene-Hexan-1-One and 1-(3-Fluoro-4-Methoxy-Phenyl)-2-Methoxymethyl-Hexan-1-One

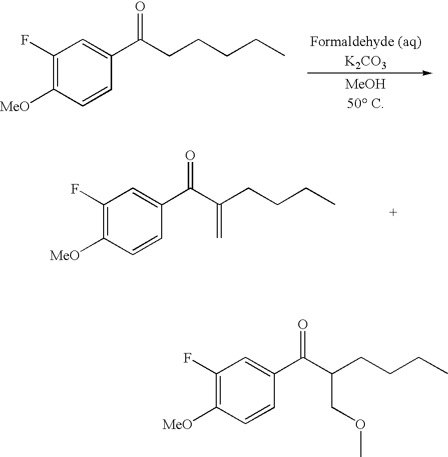

1-(3-Fluoro-4-methoxy-phenyl)-hexan-1-one (5.07 g, 22.6 mmol) is dissolved in methanol (23 mL). To the solution is added potassium carbonate (3.12 g, 22.6 mmol) and aqueous formaldehyde (2.20 g of 37%, 27.1 mmol). The slurry is heated to 50° C. stirred overnight.

The slurry is then cooled to room temperature and transferred to a separatory funnel along with toluene (23 mL) and water (23 mL). After mixing, the aqueous layer is removed and the organic washed again with water (23 mL).

The organic phase is then concentrated/azeotropically dried to a volume of about 10 mL and the solution is used in the next step.

### EXAMPLE 3

### Preparation of 2-Butyl-6-Fluoro-5-Methoxy-Indan-1-One

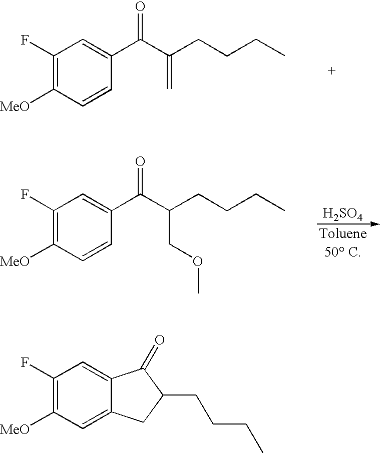

The dry toluene solution containing a mixture of vinyl ketone and methyl ether from the previous reaction (volume ˜10 mL) is diluted with toluene (40 mL) and concentrated sulfuric acid (5.0 mL) is added. This mixture is heated to 50° C. with stirring for three hours.

The mixture is then cooled to 5° C. and quenched by the slow addition of water (25 mL), keeping the temperature below 20° C.

The mixture is then transferred to a separatory funnel and the aqueous layer is removed. The organic is washed again with water (25 mL) and concentrated/azeotropically dried in vacuo. The batch is concentrated to a thick a slurry. The crude product is taken up in hot heptane (total volume of ˜20 mL, 60° C.) and the solution is allowed to cool. The product crystallizes upon cooling to room temperature. The slurry is further cooled to 0° C. for one hour and filtered, washing with cold (0° C.) 9:1 heptane/toluene (2×2.5 mL). The solid is dried under vacuum.

### EXAMPLE 4

### Preparation of 2-Butyl-4-Chloro-6-Fluoro-5-Methoxy-Indan-1-One

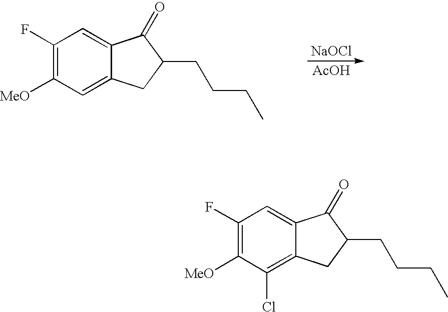

2-Butyl-6-fluoro-5-methoxy-indan-1-one (14.0 g, 59.3 mmol) was dissolved in acetic acid (177 mL) and the solution was cooled to 16° C. NaOCl (114 g of 7.57 wt % aqueous, 116 mmol) was added over 4 h, the temperature remaining at 16° C. Water (80 mL) was then added over 30 min to complete the crystallization. The slurry was filtered, washed with 1:1 HOAc:water (84 mL), then washed with water (84 mL) and finally with 2:1 MeOH:water (3×35 mL). The solid was dried under a stream of nitrogen.

### EXAMPLE 5

### Preparation of: (S)-2-Butyl-4-Chloro-6-Fluoro-2-(−3-Chloro-But-2-Enyl)-5-Methoxy-2,3,3A,7A-Tetrahydro-Inden-1-One

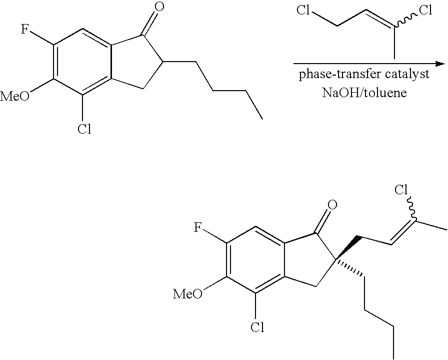

2-Butyl-4-chloro-6-fluoro-5-methoxy-indan-1-one (1.85 g, 6.83 mmol) and the phase transfer catalyst N-(4-trifluoromethylbenzyl)cinchoninium bromide (182 mg, 0.34 mmol) were combined with toluene (28 mL) and aqueous 50% NaOH (9.3 mL). The mixture was stirred under nitrogen and cooled to 10° C. 1,3-Dichloro-2-butene (1.07 g, 8.54 mmol, mixture of E and Z) was added and the mixture was rapidly stirred for 30 h. Water (28 mL) was added slowly and the temperature was allowed to rise to 20-25° C. The layers were separated and the organic layer was washed with water (9 mL) and then with 1 M aqueous citric acid (9 mL). The toluene layer was filtered and concentrated to ˜10 mL in vacuo. The solution was carried directly into the next step.

### EXAMPLE 6

### Preparation of (S)-9A-Butyl-8-Chloro-6-Fluoro-7-Methoxy-1,2,9,9A-Tetrahydro-Fluoren-3-One

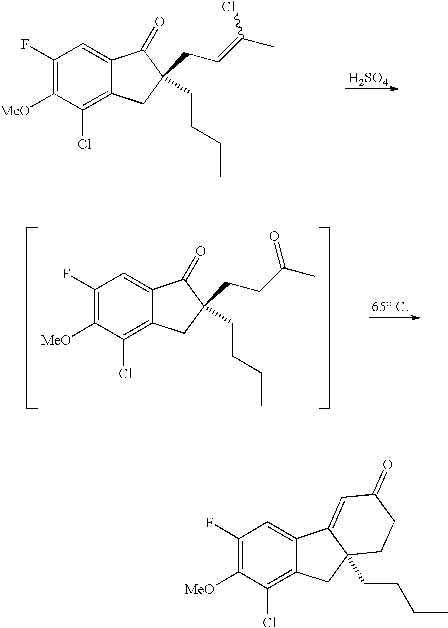

The toluene solution of (S)-2-butyl-4-chloro-6-fluoro-2-(−3-chloro-but-2-enyl)-5-methoxy-2,3,3a,7a-tetrahydro-inden-1-one was cooled in an ice bath, and to it was slowly added 7.0 mL conc H₂SO₄. The dark 2-phase mixture was stirred 1 h at 35-40° C. Water (0.22 mL) was then added and the mixture was heated to 65° C. and stirred 2 h to complete the cyclization. After cooling to 20° C., the mixture was poured slowly into 34 mL of cold (˜5° C.) water

The mixture was transferred to a separatory funnel, rinsing with a 1:1 mixture of toluene and water (3 mL). The layers were separated, the organic layer was washed with 5% NaHCO₃ solution (10 mL). The toluene was concentrated and azeotropically dried in vacuo.

### EXAMPLE 7

### Preparation of (S)-9A-Butyl-8-Chloro-6-Fluoro-7-Hydroxy-1,2,9,9A-Tetrahydro-Fluoren-3-One

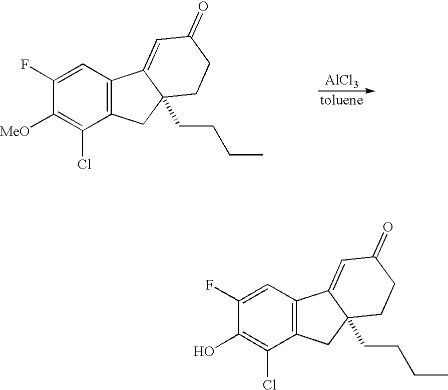

A toluene solution of (S)-9a-butyl-8-chloro-6-fluoro-7-methoxy-1,2,9,9a-tetrahydro-fluoren-3-one (170 mL containing 30.0 g, 92.9 mmol, 76% e.e.) was cooled below 5° C. under nitrogen and aluminum chloride (18.6 g, 139 mmol, 1.5 equiv.) was added. The mixture was then stirred at 40° C. for 2.5 h.

The reaction was quenched at 40 to 50° C. by slow addition of 1-propanol (29 mL). The mixture was heated at 45° C. for 1 h, then cooled to 20° C. An aqueous quench solution of citric acid, water, and 2.0 N HCl 1:8:1 (350 mL) was added, very slowly at first, with good stirring. The batch temperature was maintained between 20 and 50° C. with cooling as needed, then cooled to 20° C. when the addition was complete.

The final mixture was stirred 60 min at 20° C. and Solka Floc (7.0 g) was added. The mixture was filtered through a pad of Solka Floc to remove the precipitated racemic product, rinsing with a solution of 1-propanol (5%) in toluene (83 mL).

The filtrate was allowed to settle and the aqueous layer was removed. 1-Propanol (14 mL) was added to the organic layer and it was washed with a solution of citric acid, water, and 2.0 N HCl 1:8:1 (225 mL). The aqueous layer was removed. 1-Propanol (14 mL) was added to the organic layer and it was then washed with 0.1 N HCl (225 mL).

The final organic layer was concentrated in vacuo while adding acetonitrile, removing toluene and giving a suspension in acetonitrile with a volume of 132 mL. The mixture was heated to 55° C. to dissolve the product, then cooled to crystallize. The suspension was cooled to −20° C. for 30 min, then filtered, rinsing with −20° C. acetonitrile (55 mL). The solid was dried under vacuum at 40° C. The product crystallizes as an acetonitrile solvate which will partially release the solvent on extended drying.

### EXAMPLE 8

### Preparation of 4-Bromo-9A(S)-Butyl-8-Chloro-6-Fluoro-7-Hydroxy-1,2,9,9A-Tetrahydro-3H-Fluoren-3-One

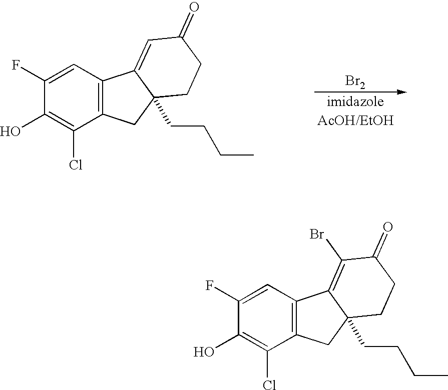

(S)-9a-Butyl-8-chloro-6-fluoro-7-hydroxy-1,2,9,9a-tetrahydro-fluoren-3-one (10.0 g, 32.4 mmol) was dissolved in acetic acid (25.0 mL) and ethanol (50.0 mL) under nitrogen. Activated carbon Darco KB-B (2.0 g) was added and the mixture was stirred at 21° C. for 60 min. The mixture was then filtered, rinsing with 2:1 ethanol/acetic acid (35.0 mL).

Imidazole (3.86 g, 56.7 mmol) was added to the combined filtrate and wash and the mixture was stirred at room temperature until the imidazole dissolved. The solution was then cooled to 0° C. Bromine (1.59 mL, 4.97 g, 31.1 mmol) was added dropwise to the stirring solution, maintaining the reaction temperature below 10° C. After 15 minutes more at 10° C., the solution was warmed to 21° C.

Darco KB-B (2.0 g) was then added. After stirring for 60 min, the mixture was filtered, rinsing with 2:1 ethanol acetic acid (35.0 mL). Water (132 mL) was then added slowly to crystallize the product. After all the water had been added, the slurry was stirred for 30 min and filtered, rinsing with 1:1 acetic acid/water (74 mL). The solid was dried under vacuum at 40° C.

Specific rotation:

solvent 70:30 MeCN:0.1% H₃PO₄ (aq)
concentration 1.0 (w/v) %
wavelength 589 nm
specific rotation −221.5 degrees

What is claimed is:

1. A process for preparing a compound of formula I: 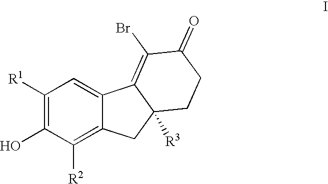
comprising reacting a compound of formula II with a brominating agent 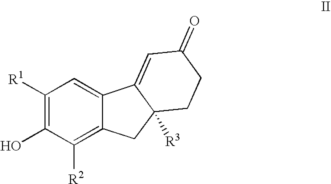
wherein R¹ is halo; R² is halo; and R³ is C₁₋₆ alkyl.

2. The process of claim 1 wherein the brominating agent is Br₂, NBS or 1,3-dibromo-5,5-dimethylhydantoin.

3. The process of claim 2 wherein the brominating agent is Br₂ and the reaction is performed in the presence of a weak base.

4. The process of claim 3 wherein the weak base is sodium acetate, potassium acetate, NaHCO₃, KHCO₃, imidazole, or pyridine wherein said imidazole and pyridine bases are optionally substituted with one or two substituents selected from halo, aryl or C₁₋₆ alkyl.

5. The process of claim 4 wherein the weak base is imidazole.

6. A compound of formula I produced by the process of claim 1.

7. The compound of claim 6 wherein R¹ is fluoro; R² is chloro; and R³ is butyl.

8. Crystalline 4-bromo-9a(S)-butyl-8-chloro-6-fluoro-7-hydroxy-1,2,9,9a-tetrahydro-3H-fluoren-3-one.

9. A process for making a compound of formula III: 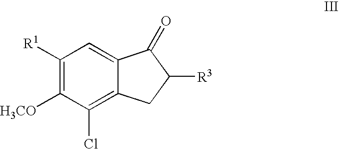
comprising reacting a compound of formula IV with a chlorinating agent 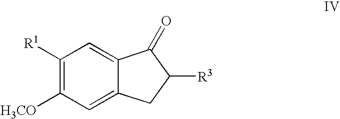
wherein R¹ is halo and R³ is C₁₋₆ alkyl.

10. The process of claim 9 wherein the chlorinating agent is NaOCl, Cl₂, t-butylhypochlorite, N-chlorosuccinimide or 1,3-dichloro-5,5-dimethylhydantoin.

11. The process of claim 9 further comprising an acid, wherein the acid is acetic acid, propionic acid, hydrochloric acid or sulfuric acid.

12. A compound of formula IV wherein R¹ is fluoro; and R³ is butyl.

13. A novel polymorphic Form A of 4-bromo-9a(S)-butyl-8-chloro-6-fluoro-7-hydroxy-1,2,9,9a-tetrahydro-3H-fluoren-3-one which is characterized by an x-ray powder diffraction pattern, collected using copper Kα radiation, with peaks at 8.6, 21.9, and 25.6 degrees (20).

14. The x-ray powder diffraction pattern of claim 13 which is further characterized by peaks at 17.5, 20.2, and 24.1 degrees (2θ).

15. The x-ray powder diffraction pattern of claim 14 which is further characterized by peaks at 14.5, 27.2, and 28.0 degrees (2θ).

16. A novel polymorphic Form A of 4-bromo-9a(S)-butyl-8-chloro-6-fluoro-7-hydroxy-1,2,9,9a-tetrahydro-3H-fluoren-3-one which is characterized by differential scanning calorimetry, at a heating rate of 10° C./min in a crimped aluminum pan, that exhibits a minor endotherm with an extrapolated onset temperature of about 172° C.

17. The novel polymorphic Form A of claim 16 which is characterized by differential scanning calorimetry, at a heating rate of 10° C./min in a crimped aluminum pan, that exhibits a major endotherm with an extrapolated onset temperature of about 177° C.

18. A novel polymorphic Form A of 4-bromo-9a(S)-butyl-8-chloro-6-fluoro-7-hydroxy-1,2,9,9a-tetrahydro-3H-fluoren-3-one which is characterized by single crystal X-ray diffraction dimensions of a=22.2, b=7.4, and c=11.1 angstroms.

19. The novel polymophic Form A of claim 18 further characterized by crystallographic angles of α=, β=111.1, and γ=90 degrees.
